(19) United States
(12) Patent Application Publication
(10) Pub. No.: US 2006/0030906 A1
(43) Pub. Date: Feb. 9, 2006

(54) Switchable and programmable electrode configuration

(76) Inventor: William J. Carroll, La Center, WA (US)

Correspondence Address: BLANK ROME LLP, 600 NEW HAMPSHIRE AVENUE, N.W., WASHINGTON, DC, 20037, US

(21) Appl. No.: 11/198,386

(22) Filed: Aug. 8, 2005

Related U.S. Application Data
(60) Provisional application No. 60/599,012, filed on Aug. 6, 2004.

(51) Int. Cl. A61N 1/18; A61N 1/32

(52) U.S. Cl. 607/67

(57) ABSTRACT

An electrode configuration for surface electrical stimulation to treat chronic and acute pain electrically re-routes current through one of a variety of possible switching means to selectively use a set of electrodes in different modes, for example, in a cross current mode or a longitudinal current mode.

### CROSS REFERENCE TO RELATED CASES

This application claims priority of U.S. Provisional Patent Application Ser. No. 60/599,012, filed Aug. 6, 2004, the entire specification of which is incorporated herein by reference.

### FIELD OF THE INVENTION

The present invention is generally related to surface electrical stimulation and, more particularly, is related to a system that uses a switchable and programmable electrode configuration during sequential stimulation

### BACKGROUND OF THE INVENTION

Surface Electrical Stimulation (also referred to as Transcutaneous Electrical Nerve Stimulation therapy or “TENS” or Neuromuscular Electrical Stimulation, “NMES”) delivers electrical pulses across the skin for various purposes. One of the main indications for Surface Electrical Stimulation (SES) is the relief of acute or chronic pain. Another type of SES (with different parameter sets and electrode configurations) is Neuromuscular Electrical Stimulation (NMES) and is used primarily but not exclusively for disuse atrophy, and or maintenance of range of motion (ROM). Other examples of Surface Electrical Stimulation are, but should not be limited to, Transcutaneous Electrical Nerve Stimulation (TENS), Neuromuscular Electrical Stimulation (NMES), Interferential Stimulation, Diadynamic Stimulation, High Volt Galvanic Stimulation (HVGS), Electro-Magnetic and Pulsed Electro-Magnetic Field Stimulation (EMF & PEMF) and Micro-current Stimulation. SES can also be utilized to effect the healing of various types of tissue, such as, bone, cartilage, connective tissue, etc.

It has been shown that sequential stimulation (stimulation of different modes or types of stimulation in a sequence) is of benefit and may provide advantages over the use of single modes of stimulation in isolation. One beneficial sequence is first using Interferential stimulation (true interferential rather than pre-modulated) to provide pain control and “quiet” the system followed by Neuromuscular Electrical Stimulation (muscle stimulation) for “working” the muscle or muscle rehabilitation.

The use of “true interferential” rather than pre-modulated interferential can have some significant benefits. True interferential produces an additive effect to the amplitude where the two circuits cross and allows deeper penetration of higher amplitude signals with less surface stimulation and potential discomfort (see e.g. FIG. 1). This can produce greater levels of stimulation and more effective pain control to deeper structures with less potential patient discomfort.

FIG. 2 shows a diagram of the current intensity. The depth of modulation can vary from 0 to 100%, depending on the direction of the currents. When two circuits intersect at 90 degrees, the maximum resultant amplitude (i) and the deepest level of modulation is halfway between the two circuits (45 degrees diagonally).

True interferential current is used with other forms of electrical stimulation in a treatment pattern or sequence. The interferential current typically has a base medium frequency alternating current between 500-20,000 Hz. An interferential current is set up between two circuits that are arranged in a cross pattern (see e.g. FIG. 1). Where the circuits superimpose, the resultant beat frequency will be the difference between the frequencies of the two circuits and the amplitude will be additive and greater than either circuit alone. The range of the beat frequency achieved is usually between 1-250 beats per minute. Multiple levels of stimulation can be treated depending on the electrode pairing combination and modulation pattern selected. The range of output can be from 0-100 volts per circuit, depending on the patient's needs. The amplitude can be modulated in the respective circuits to increase the area of targeted stimulation. This type of current (interferential) provides improved directional control, decreased accommodation/habituation and depth of penetration in comparison to other types of standard surface stimulation systems. This can also apply to standard implantable stimulation systems and their accompanying surgical leads.

The inventor has determined that compliance with instructions to manually change electrode configuration is very low. Consequently, manufacturers of devices capable of sequential styles of stimulation have historically used pre-modulated interferential which does not require a change in configuration, but at the same time does not give the full benefit of true interferential stimulation.

Thus, there is a need in the industry for improvements in apparatus and methods for applying electrical stimulation.

### SUMMARY OF THE INVENTION

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In an embodiment, a switchable and programmable electrode configuration is applied during a process of sequential stimulation (a process of stimulation that includes more than one type or sequence of electrical stimulation). Previously, patients were required to manually change the electrode configuration (but not necessarily the electrode placement) from a cross pattern to a longitudinal pattern, which is inconvenient. In preferred embodiments, the switchable configuration disclosed herein allows switching of electrodes between a plurality of predetermined connection patterns, such as, for example, between a longitudinal pattern and a cross pattern.

Additional features and advantages of the invention will be set forth in the description which follows, and in part will be apparent from the description, or may be learned by practice of the invention. The advantages of the invention will be realized and attained by the structure particularly pointed out in the written description and claims hereof as well as the appended drawings.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views:

FIG. 1 is a perspective drawing showing an interferential pattern area;

FIG. 2 is a drawing of an interferential current density pattern;

FIG. 3 is a drawing of a true interferential current stimulation electrode configuration;

FIG. 4 is a drawing of an electrode configuration for pre-modulated interferential current stimulation using a longitudinal pattern appropriate for NMES;

FIG. 5 is a schematic drawing of an embodiment of the invention;

FIG. 6 is a schematic drawing of an embodiment of the invention;

FIG. 7 is a perspective drawing of electrode placement for pain control around the knee; and

FIG. 8 is a perspective drawing of electrode placement for muscle maintenance near the knee.

### DETAILED DESCRIPTION OF THE INVENTION

This disclosure provides and discloses exemplary embodiments. In particular, the specification discloses one or more embodiments that incorporate the features of the invention. The embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, persons skilled in the art may effect such feature, structure, or characteristic in connection with other embodiments whether or not explicitly described.

Embodiments may be implemented in hardware, firmware, software, or any combination thereof. Embodiments of the invention may also be implemented as instructions stored on a machine-readable medium, which may be read and executed by one or more processors. A machine-readable medium may include any mechanism for storing or transmitting information in a form readable by a machine (e.g. a computing device). For example, a machine-readable medium may include read only memory (ROM); random access memory (RAM); hardware memory in PDAs, mobile telephones, and other portable devices; magnetic disk storage media; optical storage media; flash memory devices; electrical, optical, acoustical, or other forms of propagated signals (e.g. carrier waves, infrared signals, digital signals, analog signals, etc.), and others. Further, firmware, software, routines, instructions, may be described herein as performing certain actions. However, it should be appreciated that such descriptions are merely for convenience and that such actions in fact result from computing devices, processors, controllers or other devices executing the firmware, software, routines, instructions, etc.

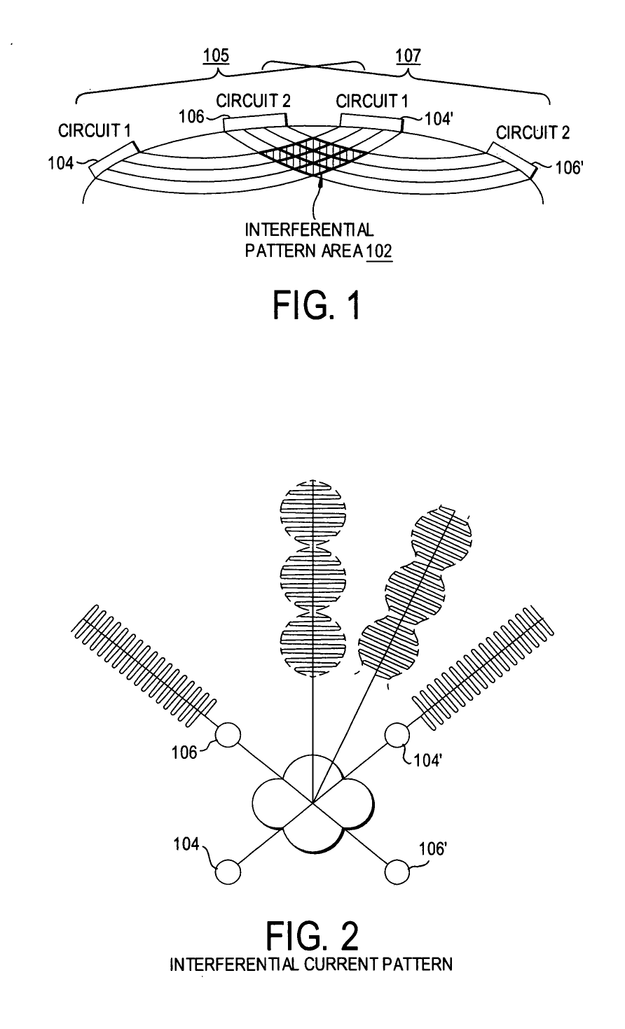

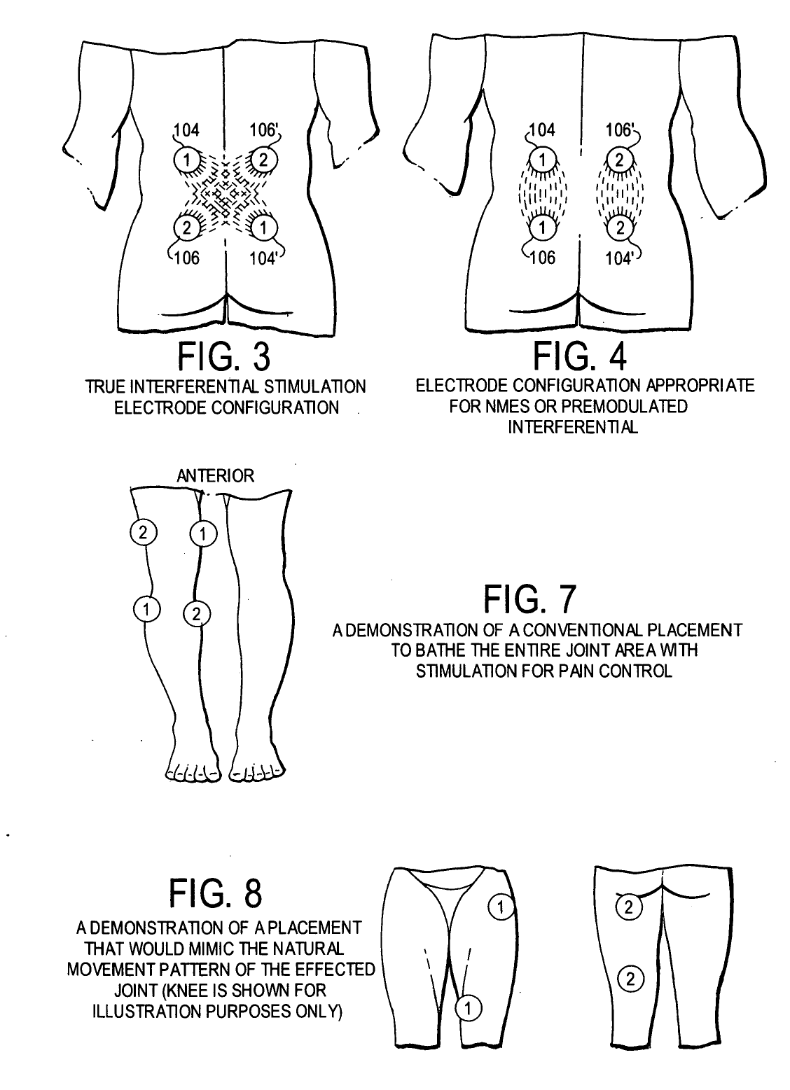

An exemplary, preferred embodiment includes starting with a first portion of a treatment protocol with 15-30 minutes of true interferential stimulation, which requires a cross pattern of at least two circuits. FIG. 1 shows an interferential pattern area 102 formed at the cross pattern intersection of electrodes 104 and 104′ defining a first circuit 105 and electrodes 106 and 106′ defining a second circuit 107. FIG. 2 shows the interferential current pattern generated by the arrangement of FIG. 1. FIG. 3 shows electrodes 104 and 104′ and 106 and 106′ applied across the spinal area. A second treatment protocol, for example, NMES, is then applied. This protocol requires a longitudinal placement of electrodes along the length of the target muscle. Referring to FIG. 4, an exemplary placement is shown with a first circuit using electrodes 104 and 106, and a second circuit using electrodes 104′ and 106′.

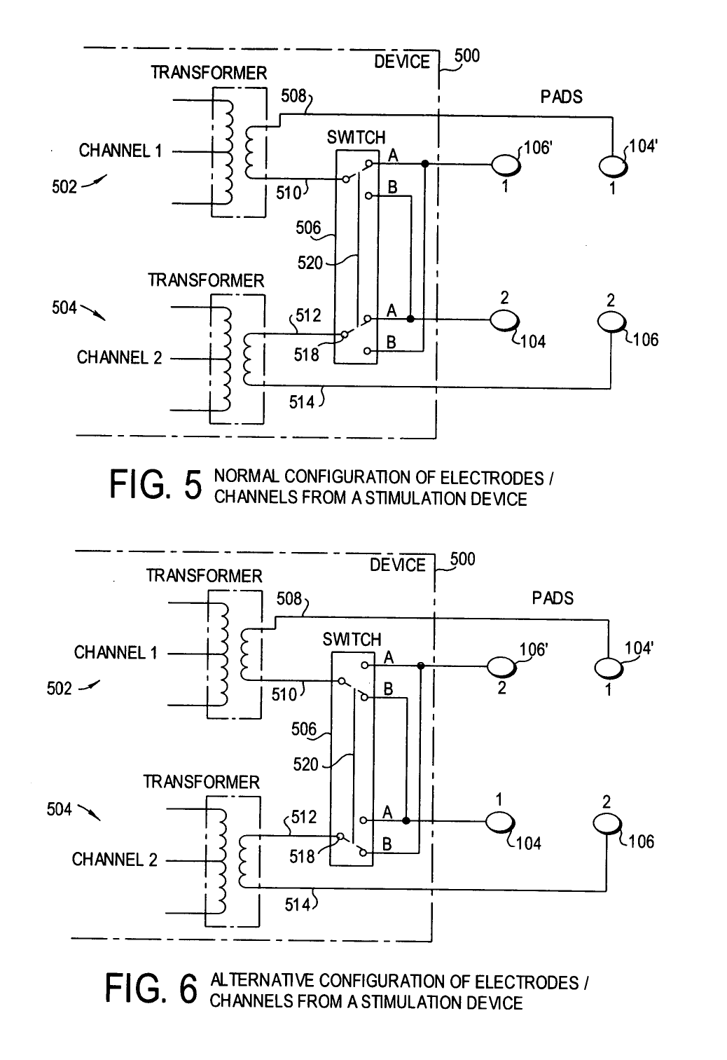

FIGS. 5 and 6 demonstrate an exemplary embodiment of the invention that uses a mechanical (hardware) switch or relay to re-route the current from two of the electrodes (one from each channel). As shown in FIGS. 5 and 6, a device 500 incorporates a first channel circuit 502 and a second channel circuit 504. Outputs 508 and 510 of circuit 502 are connected respectively to electrode 104′ and to an input 516 of switch 506.

Input 516 of switch 506 is selectively connected to one of outputs A and B associated with input 516, depending on the state of switch 506. Outputs A and B are connected to electrodes 106′ and 104 respectively. Second channel circuit 504 has outputs 512 and 514, connected respectively to input 518 of switch 506 and to electrode 106. Input 518 of switch 506 is connected either to electrode 104 or to electrode 106′, depending on the state of switch 506.

In this embodiment, switch 506 is shown schematically as a double pole, double throw switch. However, it will be understood that the scope of the invention is not limited to any particular type of switch. The functionality of switch 506 can be implemented either as a mechanical switch, a solid state or electronic switch, or in any other known manner that produces similar results in terms of the application of electrical stimulation in the desired patterns.

As can be seen, when switch 506 is in the first state (inputs connected to the A outputs), as shown in FIG. 5, a longitudinal pattern electrode configuration is provided. In a second state of switch 506, as shown in FIG. 6, a cross pattern electrode configuration is provided.

Other desirable embodiments utilize electronic circuitry to switch the electrode configuration and have the advantage of smaller size, but provide similar functionality. The electronic circuitry may vary the state of the electrode connections under control of an automatic program or processor to provide an appropriate automated sequence of stimulation. Switch 506 has a control means 520 for actuating the switch. Control means 520 may be any desired mechanism for controlling the state of the switch or switch equivalent used in the circuit. For example, without limitation, control means 520 may be a manual activation by a user or clinician, a timer, an electronic control, or a microprocessor control responding to a program for applying a predetermined sequence of stimulations.

A switchable and programmable electrode configuration will greatly benefit patients suffering from pain and muscle dysfunction by increasing their compliance to the prescribed protocol while obtaining the added benefits of true interferential stimulation.

Another use for a switchable electrode configuration is for incorporation into garments that include multiple electrodes (garment not shown). One such garment is provided for the knee to activate surface electrical stimulation surrounding a joint or area of pain (see FIG. 7), and then switch to a different configuration that would facilitate patterned or reciprocal stimulation of the agonist (Quadriceps) and antagonist (Hamstrings) muscle groups (see FIG. 8).

While various embodiments of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalents.

What is claimed is:

1. An apparatus that employs a switchable and programmable electrode configuration during sequential surface electrical stimulation.

2. The apparatus according to claim 1, wherein the sequential surface electrical stimulation includes at least one true interferential stimulation component and at least one pre-modulated stimulation component.

3. The apparatus according to claim 1, wherein the electrode configuration may be sequentially switched between a cross pattern of surface electrodes for at least two circuits and a longitudinal pattern of the surface electrodes for the at least two circuits.

4. A method for switching and programming surface electrode stimulation to include a cross pattern for interferential current stimulation and a longitudinal pattern for pre-modulated stimulation.

5. A method of providing selective surface electrical stimulation, comprising:
providing a first stimulation circuit, connected to at least two first circuit electrodes in a first state;
providing a second stimulation circuit, connected to at least two second circuit electrodes in the first state;
using a switch means, selectively changing circuit connections in a second state so that the first stimulation circuit is connected to at least one second circuit electrode and the second stimulation circuit is connected to at least one first circuit electrode.

6. The method of claim 5, wherein the switch means is an electronic switch.

7. The method of claim 5, wherein the switch means is a mechanical switch.

8. The method of claim 5, wherein the switch means performs the function of a double pole double throw switch.

9. The method of claim 5, wherein the switch means operates under manual control.

10. The method of claim 5, wherein the switch means operates under a programmed electronic control.

11. The method of claim 5 wherein in one of the first state and the second state, the electrodes are connected to apply a cross current, and in the other of the first state and the second state, the electrodes are connected to apply a longitudinal current.
